(19) United States
(12) Patent Application Publication
(10) Pub. No.: US 2014/0271855 A1
(43) Pub. Date: Sep. 18, 2014

(54) SOVAPREVIR TABLETS

(71) Applicant: Achillion Pharmaceuticals Inc., New Haven, CT (US)

(72) Inventors: Jennifer Hsing-Chung Chu, Ivoryton, CT (US); Gautam Shah, Westport, CT (US); Avinash Phadke, Branford, CT (US)

(73) Assignee: Achillion Pharmaceuticals Inc., New Haven, CT (US)

(21) Appl. No.: 14/208,195

(22) Filed: Mar. 13, 2014

Related U.S. Application Data
(60) Provisional application No. 61/790,645, filed on Mar. 15, 2013.

(51) Int. Cl. A61K 9/28; A61K 9/20; A61K 38/05

(52) U.S. Cl. CPC A61K 9/28; A61K 38/05; A61K 9/2054; A61K 9/2027; A61K 9/2059; A61K 9/2013; A61K 9/2009; A61K 9/2018
USPC 424/474; 546/153; 514/21.91

(57) ABSTRACT

The disclosure includes tablet cores comprising Sovaprevir and a crystal growth inhibitor selected from hydroxypropyl methyl cellulose (HPMC), HPC (hydroxypropyl cellulose), hypromellose acetate succinate (HPMCAS), polyvinyl pyrrolidone (PVP), copovidone (PVP-VA), a copolymer of methacrylic acid and ethyl acrylate, or any combination of the foregoing, wherein ratio of Sovaprevir to crystal growth inhibitor is from about 40:60 (w/w) to about 60:40 (w/w). The disclosure further includes film coated tables. The disclosure also includes methods of treating HCV with the tablets described herein.

### CROSS REFERENCE TO RELATED APPLICATION

This application claims priority from U.S. Provisional application No. 61/790,645 filed Mar. 15, 2013, which is hereby incorporated by reference in its entirety.

### FIELD OF THE DISCLOSURE

Sovaprevir (ACH-0141625) tablets and tablet cores are provided by this disclosure.

### BACKGROUND

Sovaprevir is a hepatitis C virus NS3 protease inhibitor, effective for treating HCV infection in humans.

[chemical structure]

Sovaprevir has challenging physical and chemical properties relevant for preparation of a solid dosage form. Sovaprevir is sufficiently soluble in its amorphous form, but has at least six crystal polymorphs which have altered pharmacokinetics and reduced solubility compared with the amorphous form. Sovaprevir has a tendency to form crystalline forms in storage under a variety of conditions. Sovaprevir crystalline forms exhibit differences in physical and chemical properties relative to the amorphous form making their presence in Sovaprevir dosage forms undesirable.

The physical and chemical characteristics of a pharmaceutical dosage form of a drug contribute to the drug's activity, manufacturing, utility, and efficacy. These include content and mass uniformity, solubility, bioavailability, particle size and shape, chemical and physical stability, dissolution rate, and bioavailability. In addition the mixtures of crystal polymorphs can cause variability in processing conditions, such as density and flow properties.

The physical and pharmacokinetic properties of an API can often be improved by judicious choice of excipients such as lubricants, disintegrants, fillers, and crystallization inhibitors. The manner in which these ingredients are combined and processed into final solid dosage forms as tablets, capsules, lozenges, and so forth can also result in improved response to the dosed drug.

Stable Sovaprevir dosage forms having consistent and desirable physical and chemical properties are needed. This disclosure provides dosage forms that meet this need and have additional advantages which are described in the disclosure.

### SUMMARY

The disclosure provides a Sovaprevir immediate release composition, containing an amount of Sovaprevir from about 100 mg to about 400 mg Sovaprevir; wherein the composition provides a mean AUC₀-₇₂ of about 150 ng·hr/ml to about 2500 ng·hr/ml; a mean C. of about 20 ng/ml to about 750 ng/ml; and a mean T. of about 0.5 to about 5.0 hr.

The disclosure also provides a Sovaprevir tablet, comprising Sovaprevir, and a crystal growth inhibitor selected from hydroxypropyl methyl cellulose also known as hypromellose (HPMC), hydroxypropyl cellulose (HPC), hypromellose acetate succinate (HPMCAS), polyvinyl pyrrolidone (PVP), copovidone (PVP-VA), a copolymer of methacrylic acid and ethyl acrylate, or any combination of the foregoing, wherein ratio of Sovaprevir to crystal growth inhibitor is from about 40:60 (w/w) to about 60:40 (w/w).

### BRIEF DESCRIPTION OF THE DRAWINGS

FIG. 1. Process diagram for manufacture of Sovaprevir tablets.

### DETAILED DESCRIPTION

### Tablet Composition

A new Sovaprevir tablet dosage form has been developed. While Sovaprevir tablets may contain a range of the active agent, Sovaprevir, tablets containing 100 mg, 200 mg, 250 mg, and 300 mg of Sovaprevir are exemplified.

The disclosure includes Sovaprevir formulations in which Sovaprevir comprises about 15% to about 40% by weight of the tablet core and the crystal growth inhibitor comprises about 15% to about 50% by weight of the tablet core. In certain embodiments the Sovaprevir comprises about 20% to about 30% by weight of the tablet core and the crystal growth inhibitor comprises about 20% to about 35% by weight of the tablet core.

Suitable excipients for the tablet core include hydroxypropyl methyl cellulose (HPMC), such as METHOCEL ES, as a crystal growth inhibitor, silicified microcrystalline cellulose (Prosolv SMCC 90) as a filler/diluent, croscarmellose sodium (Ac-Di-Sol) as a disintegrant and magnesium stearate as a lubricant. The ratio of Sovaprevir: HPMC may be from about 10:90 (w/w) to from 90:10 (w/w), or from about 30:70 (w/w) to about (70:30), or from about 40:60 (w/w) to about 60:40 (w/w) or more particularly about 50:50 (w/w). A number of other compounds can serve as effective Sovaprevir crystal growth inhibitors including: cellulose polymers HPC (hydroxypropyl cellulose), hydroxypropyl methyl cellulose (HPMC) also known as hypromellose, (such as Hypromellose 2910 USP), HPMCAS (hypromellose acetate succinate), the synthetic polymers, PVP (polyvinyl pyrrolidone), PVP-VA (copovidone) and polymethacrylate based polymers. Ratios are given as a weight percentage. A 30:70 w/w ratio for Sovaprevir to crystal growth inhibitor means weight of Sovaprevir is 30% of the total of the weight of Sovaprevir plus crystal growth inhibitor.

A crystal growth inhibitor is an agent used to improve the stability of a pharmaceutical formulation by preventing the crystal growth of a pharmaceutical agent. The crystal growth inhibitor is present in the composition at a concentration sufficient to significantly reduce the formation of crystals in a Sovaprevir composition over time relative to a Sovaprevir composition that does not contain the crystal growth inhibitor.

The disclosure includes tablet cores and tablets containing a lubricant. For example the lubricant may be stearic acid, magnesium stearate, glyceryl behenate, calcium stearate, sodium stearyl fumarate, sodium lauryl sulfate, magnesium lauryl sulfate, or sodium benzoate. The lubricant may be present in the tablet core or coated tablet in an amount (% w/w) from about 0.1% to about 2% or from about 0.1% to about 1.0%, or from about 0.25% to about 0.5%, or about 0.375%. Magnesium stearate is an exemplary lubricant.

The disclosure includes tablet cores and coated tablets containing a filler. For example the filler may be lactose monohydrate, anhydrous lactose, mannitol, dextrose, glucose, microcrystalline cellulose, starch, calcium carbonate, dicalcium phosphate, or magnesium carbonate. The filler may be present in the tablet core or coated tablet in an amount (% w/w) from about 20% to about 70%, or from about 40% to about 50%, or about 44.75%. Prosolv SMCC 90, a type of silicified microcrystalline cellulose, is an exemplary filler.

The disclosure includes tablet cores and tablets containing a disintegrant. For example the disintegrant may be croscarmellose sodium, polyvinylpyrrolidone, microcrystalline cellulose, alginic acid, sodium alginate or sodium starch glycolate. The disintegrant may be present in the tablet core or coated tablet in an amount (% w/w) from about 1% to about 20%, or about 5%.%. About 5% Ac-Di-Sol is an exemplary disintegrant.

This disclosure includes tablets that have a non-functional film coating.

This disclosure includes the following particular embodiments.

A Sovaprevir immediate release composition, comprising an amount of Sovaprevir from about 100 mg to about 400 mg Sovaprevir; wherein the composition provides an AUC₀-₇₂ of about 150 ng·hr/ml to about 2500 ng·hr/ml; a mean C. of about 20 ng/nl to about 750 ng ml; and a mean T. of about 0.5 to about 5.0 hr.

A 200 mg Sovaprevir composition providing an AUC₀-₇₂ of about 300 ng·hr/ml to about 700 ng·hr/ml a mean C. of about 40 ng/ml to about 170 ng/ml; and a mean T. of about 1.5 to about 3.7 hr.

A Sovaprevir tablet core, comprising Sovaprevir and a crystal growth inhibitor selected from hydroxypropyl methyl cellulose (HPMC), HPC (hydroxypropyl cellulose), hypromellose acetate succinate (HPMCAS), polyvinyl pyrrolidone (PVP), and copovidone (PVP-VA), or any combination of the foregoing, wherein ratio of Sovaprevir to the crystal growth inhibitor is from about 40:60 (w/w) to about 60:40 (w/w).

A Sovaprevir tablet core as in any of the embodiments previously described, wherein the crystal growth inhibitor is HPMC and the ratio of Sovaprevir to crystal growth inhibitor is about 50:50 (w/w).

A Sovaprevir tablet core as in any of the embodiments previously described, wherein the crystal growth inhibitor is a combination of HPMC and a second crystal growth inhibitor and the ratio of Sovaprevir to crystal growth inhibitor is about 50:50 (w/w).

A Sovaprevir tablet core as in any of the embodiments previously described, wherein the tablet core also comprises a filler, a disintegrant, and a lubricant.

In certain embodiments the disintegrant is croscarmellose sodium, crospovidone, sodium starch glycolate, or a combination of the foregoing.

In certain embodiments the lubricant is calcium stearate, magnesium stearate, glyceryl monostearate, glyceryl behenate, stearic acid, mineral oil, talc, or a combination of the foregoing.

In certain embodiments the filler is microcrystalline cellulose, silicified microcrystalline cellulose, ethyl cellulose, lactose, or a combination of the foregoing.

In certain embodiments the filler is microcrystalline cellulose or silicified microcrystalline cellulose or a combination thereof, the disintegrant is croscarmellose sodium, and the lubricant is magnesium stearate.

A Sovaprevir tablet core, wherein the tablet core comprises about 20% to about 30% (weight %) Sovaprevir, about 20% to about 30% HPMC, about 40% to about 60% silicified microcrystalline cellulose, about 1% to about 10% croscarmellose sodium, and about 0.1% to about 1% magnesium stearate.

A coated Sovaprevir tablet comprising the Sovaprevir tablet core of any or the previous embodiments and an immediate release coating.

The Sovaprevir tablet core of of any or the previous embodiments, wherein the tablet core exhibits a dissolution profile after combining the tablet with 900 mL of 0.5% SLS in DI water at 37° C. according to USP 36 <711> (paddle) at a speed of 75 rpm, wherein at least 60% of the total amount of Sovaprevir is released after 10 minutes.

The coated Sovaprevir tablet comprising the Sovaprevir tablet core of any of the previous embodiments and an immediate release coating, wherein the tablet core exhibits a dissolution profile after combining the tablet with 900 mL of 0.5% SLS in DI water at 37° C. according to USP 36 <711> (paddle) at a speed of 75 rpm, wherein at least 40% of the Sovaprevir is released after 10 minutes.

A Sovaprevir tablet core of any of the previous embodiments, wherein 90% of the total amount of Sovaprevir is released after 30 minutes.

A coated Sovaprevir tablet of any of the previous embodiments, wherein the tablet exhibits a dissolution profile after combining the tablet with 900 mL of 0.5% SLS in DI water at 37° C. according to USP 36 <711> (paddle) set at 75 rpm, wherein at least 70% of the total amount of Sovaprevir is released after 30 minutes.

A Sovaprevir tablet core of any of the previous embodiments, wherein at least 95% of the total amount of Sovaprevir released after 60 minutes.

A coated Sovaprevir tablet of any of the previous embodiments, wherein at least 90% of the Sovaprevir is released after 60 minutes.

Embodiments may be combined only so long as a stable tablet or tablet core results. “A combination of any of the foregoing” only includes combinations that result in a stable tablet or tablet core.

### Tablet Manufacture

The tablet dosage form for Sovaprevir uses common tableting excipients, process parameters and conventional equipment. The Sovaprevir tablets use a common blend that is manufactured via dry granulation such as roller compaction followed by milling. Blending/milling and tableting equipment and processes are used. Common film coating equipment and processes are used to coat the tablet cores.

Sovaprevir blends can be prepared by a variety of granulation processes, including spray drying, solvent wet granulation, aqueous wet granulation and dry granulation using roller compaction, however dry granulation using roller compaction efficiently produces Sovaprevir tablet blends with desirable bulk density and flow properties.

The manufacturing process of Sovaprevir tablets is optimized through evaluation of blend and tablet physical properties including bulk and tap density measurement, flow analysis, screen analysis, and uniformity of the blend; weight, thickness, hardness, friability, potency, disintegration, dissolution and content uniformity tests on the tablet cores and tablets.

The disclosure provides a process for manufacturing Sovaprevir tablets. In one embodiment the process includes the following steps.

First, a half portion of hydroxypropyl methyl cellulose is charged into a blender, such as a V-blender or bin blender, followed by addition of Sovaprevir and then the remaining half portion of hydroxypropyl methyl cellulose, and then blending the materials.

Magnesium stearate may be screened to break up any agglomerates such as through a 20 mesh screen. The screened magnesium stearate is added to the blender containing the Sovaprevir/crystal growth inhibitor blend and blended for several minutes. The Sovaprevir/crystal growth inhibitor/magnesium stearate blend from the blender is discharged.

The material discharged from the blender is roller compacted to form roller compacted ribbons or compacts. The roller compacted material is then passed through a mill, such as an oscillating mill, impact mill, or screening mill. For example a QUADRO COMIL (Quadro Engineering, Ontario, Canada) equipped with a 20 mesh screen may be used. The milled material is collected and then charged into a blender. Silicified microcrystalline cellulose (Prosolv SMCC 90) and croscarmellose sodium (Ac-Di-Sol) are added and the materials are blended.

Additional magnesium stearate may be screened to break up any agglomerates such as through a 20 mesh screen into the blender and blended several minutes.

The contents of the blender are then discharged into an appropriately labeled container, double lined with polyethylene bags with a desiccant between the two layers of bags.

Tablet cores are then formed on a rotary tablet press. Tablet cores may be coated in a film coater.

### Pharmacokinetic Properties

A single 200 mg dose of Sovaprevir was administered to human subjects. This provided a mean AUC₀-₇₂ of about 300 ng·hr/m1 to about 700 ng·hr/ml, or more preferably about 400 ng·hr/ml to about 600 ng·hr/ml, or about 500 ng·hr/mL; a mean AUC₀-_{∞} of about 300 ng˜hr/ml to about 700 ng·hr/ml, or more preferably about 400 ng·hr/ml to about 600 ng·hr/ml, or about 500 ng·hr/ml; mean Cₘₐₓ of about 40 ng/ml to about 170 ng/ml, or more preferably about 80 ng/ml to about 140 ng/ml, or about 110 ng/ml; and a mean Tₘₐₓ of about 1.5 to about 3.7 hr, or more preferably about 2.1 to about 3.1 hr, or about 2.6 hr.

### EXAMPLES

### General Analytical Procedures

### Assay and Uniformity of Dosage Units (HPLC)

The content uniformity of Sovaprevir tablets are determined by reverse-phase chromatography with detection by UV absorbance at 254 nm. The mobile phase is 0.01 M phosphate buffer, pH 3.0, and gradient elution with acetonitrile is performed. The HPLC column is Waters Symmetry Shield RP18, 4.6×150 mm, 3.5 μm, or equivalent. The column temperature is 30° C. and sample temperature is ambient (25° C.). Flow rate is 0.75-1.0 mL/min. and injection volume is 10 μL. The Sovaprevir content is determined with an external standard.

### Dissolution (USP <711>)

Methods USP 34 <711> or USP 36 <711> are used to determine the dissolution characteristics of Sovaprevir tablets. The dissolution test is performed in 900 mL of 0.5% SLS (Sodium Lauryl Sulfate) in DI water at 37° C. using USP Apparatus 2 (paddles) set at 75 rpm. The samples are quantified by HPLC using Phenomenex Luna C18(2), 5 μm, 4.6×150 mm, column with a mobile phase of 25:75 phosphate buffer:methanol at a flow rate of 1.2 mL/min. UV detection is at a wavelength of 225 nm.

### Example 1

### Sovaprevir Tablet Compositions

Three dosage strengths for Sovaprevir tablet cores are exemplified, 100 mg, 200 mg, and 250 mg. The 100 mg tablet core size is 7/16 inch standard round concave and the theoretical weight is 400 mg. The 200 mg tablet core size is 9/16 inch standard round concave and the theoretical weight is 800 mg. A 200 mg modified oval tablet core, 0.34 inches×0.70 inches is also included. The 250 mg tablet core is a 0.3652 inch×0.7480 inch modified oval tablet core with a theoretical weight of 1000 mg. The 300 mg tablet core is a modified oval tablet core with dimensions 0.3990 inches×0.7550 inches. All dimensions refer to tooling size, the actual dimensions of the finished tablet cores may differ slightly. All tablet cores are mottled off-white to yellow in appearance. The formulation for the 100 mg, 200 mg, and 250 mg tablet is the same. A common blend is used to manufacture 100, 200, and 250 mg strength tablets by using appropriate fill weights.

The compositions of the 100 mg, 200 mg, and 250 mg tablet cores are listed in TABLE 1.

TABLE 1

| | | | Quantity per Tablet (mg) | | |
|---|---|---|---|---|---|
| | | Amount | 100 mg | 200 mg | 250 mg |
| Components | Function | (%) | Tablet | Tablet | Tablet |
| Sovaprevir | Active | 25.0 | 100.0 | 200.0 | 250.0 |
| Hypromellose | Crystal | 24.875 | 99.5 | 199.0 | 248.75 |
| 2910 (Methocel | Growth | | | | |
| E5) USP | Inhibitor | | | | |
| Silicified | Filler | 44.75 | 179.0 | 358.0 | 447.5 |
| Microcrystalline | | | | | |
| Cellulose (ProSolv | | | | | |
| SMCC90) USP, | | | | | |
| EP | | | | | |
| Croscarmellose | Dis- | 5.00 | 20.0 | 40.0 | 50.0 |
| sodium(Ac-DiSol) | integrant | | | | |
| NF, EP | | | | | |
| Magnesium | Lubricant | 0.375 | 1.5 | 3.0 | 3.75 |
| Stearate Non- | | | | | |
| Bovine | | | | | |
| Total | | 100.00 | 400.00 | 800.00 | 1000.00 |

### Example 2

### Alternate Tablet Formulation

Another example of tablet formulation is provided in TABLE 2. This formulation is shown for 100 mg, 200 mg, and 250 mg tablets.

TABLE 2

| | | | Quantity per Tablet (mg) | | |
|---|---|---|---|---|---|
| | | Amount | 100 mg | 200 mg | 250 mg |
| Components | Function | (%) | Tablet | Tablet | Tablet |
| Sovaprevir | Active | 25.0 | 100.0 | 200.0 | 250.0 |
| Hypromellose | Crystal | 25.0 | 100.0 | 200.0 | 250.0 |
| 2910 (Methocel | Growth | | | | |
| E5) USP | Inhibitor | | | | |
| Microcrystalline | Filler | 45.0 | 180.0 | 360.0 | 450.0 |
| Cellulose (Avicel | | | | | |
| PH 102) NF, EP, | | | | | |
| JP | | | | | |
| Sodium Starch | Dis- | 4.6 | 18.4 | 36.8 | 46.0 |
| Glycolate | integrant | | | | |
| (Explotab) NF, | | | | | |
| EP | | | | | |
| Magnesium | Lubricant | 0.4 | 1.6 | 3.2 | 4.0 |
| Stearate Non- | | | | | |
| Bovine | | | | | |
| Total | | 100.00 | 400.00 | 800.00 | 1000.00 |

### Example 3

### Film Coated Tables

Tablet cores in TABLES 1 and 2 can be film coated. Examples of these coated tablets are shown in TABLE 3. This example uses the coating Opadry White 85F18422, but other coatings could be substituted.

TABLE 3

| | Quantity per Tablet (mg) | | |
|---|---|---|---|
| | 100 mg | 200 mg | 250 mg |
| Components | Tablet | Tablet | Tablet |
| Tablet Core | 400.0 | 800.0 | 1000.0 |
| Film Coating | 12.0 | 24.0 | 30.0 |
| (Opadry White | | | |
| 85F18422) | | | |
| Total Tablet | 412.0 | 824.0 | 1030.0 |

### Example 4

### Human Sovaprevir Pharmacokinetic Parameters

The pharmacokinetic parameters shown in TABLE 4 were measured in humans following administration of a single Sovaprevir tablet dose.

TABLE 4

| | | Cmax | Tmax | AUC(0-72) | AUC(0-∞) |
|---|---|---|---|---|---|
| Dose | | (ng/mL) | (hr) | (ng · hr/mL) | (ng · hr/mL) |
| 200 mg | Mean | 109 | 2.61 | 489.9 | 500.0 |
| | SD | 62.1 | 1.08 | 190.0 | 192.4 |
| | % CV | 57 | 41 | 38 | 38 |
| | N | 23 | 23 | 23 | 23 |
| 250 mg | Mean | 172 | 3.40 | 735.6 | 748.8 |
| | SD | 124 | 1.41 | 380.3 | 386.9 |
| | % CV | 72 | 41 | 51 | 51 |
| | N | 23 | 23 | 23 | 23 |
| 300 mg | Mean | 333 | 3.39 | 1301 | 1314 |
| | SD | 199 | 0.782 | 779.0 | 785.4 |
| | % CV | 59 | 23 | 59 | 59 |
| | N | 23 | 23 | 23 | 23 |
| 100 mg | Mean | 61.9 | 4.20 | 220 | 228 |
| (Fed) | SD | 24.3 | 0.837 | 67.3 | 73.3 |
| | % CV | 39.3 | 19.9 | 30.7 | 32.1 |
| | N | 23 | 23 | 23 | 23 |
| 100 mg | Mean | 67.2 | 1.58 | 288 | 302 |
| (Fasted) | SD | 55.4 | 1.28 | 181 | 182 |
| | % CV | 82.5 | 80.9 | 62.9 | 60.4 |
| | N | 23 | 23 | 23 | 23 |

### Example 5

### Coated 250 MG Tablets

Purified water USP/EP (660 g) was weighed into a stainless steel container and was mixed at an appropriate speed to create a vortex. Opadry White 85F18422 (90.0 g) was added to the stirred water and the resulting suspension was mixed for 1 hour. A Compulab 24 Coater was charged with the suspension and the spray was adjusted. The Compulab 24 Coater with 15 inch pan was then charged with 250 mg Sovaprevir tablets (1500 g) and the pan speed was set at 21 rpm. The airflow and heat were turned on to achieve an exhaust temperature of 50° C. The tablets were then coated with the Compulab 24 Coater. Tablets were removed during the process to check appearance and weight gain. When coating was complete, the heat was turned off and the tablets were allowed to dry for at least 5 minutes. Average uncoated tablet weight was 1.0674 g; average coated tablet weight was 1.0956 g; average % weight gain was 2.64%.

### Example 6

### Coated 300 MG Tablets

Purified water USP/EP (1100 g) was weighed into a stainless steel container and was mixed at an appropriate speed to create a vortex. Opadry White 03K18416 (150.0 g) was added to the stirred water and the resulting suspension was mixed for 1 hour. A Compulab 24 Coater was charged with the suspension and the spray was adjusted. The Compulab 24 Coater with 15 inch pan was then charged with 300 mg Sovaprevir tablets (1500 g) and the pan speed was set at 15 rpm. The airflow and heat were turned on to achieve an exhaust temperature of 48° C. The tablets were then coated with the Compulab 24 Coater. Tablets were removed during the process to check appearance and weight. When coating was complete, the heat was turned off and the tablets were allowed to dry for at least 5 minutes. Average uncoated tablet weight was 1.196 g; average coated tablet weight was 1.230 g; average % weight gain was 2.84%.

### Example 7

### Coated 200 MG Tablets

Purified water USP/EP (1100 g) was weighed into a stainless steel container and was mixed at an appropriate speed to create a vortex. Opadry White 03K18416 (150.0 g) was added to the stirred water and the resulting suspension was mixed for 1 hour. A Compulab 24 Coater was charged with the suspension and the spray was adjusted. The Compulab 24 Coater with 15 inch pan was then charged with 200 mg Sovaprevir tablets (1500 g) and the pan speed was set at 12 rpm. The airflow and heat were turned on to achieve an exhaust temperature of 48° C. The tablets were then coated with the Compulab 24 Coater. Tablets were removed during the process to check appearance and weight gain. When coating was complete, the heat was turned off and the tablets were allowed to dry for at least 5 minutes. Average uncoated tablet weight was 0.821 g; average coated tablet weight was 0.837 g; average % weight gain was 1.95%.

### Example 8

### Stability of Amorphous Sovaprevir

TABLE 5 shows the stability of the amorphous form of Sovaprevir upon storage of the Sovaprevir plus excipient for the specified time under the specified conditions. Analysis of the initial API showed it was amorphous in nature. Upon storage of Sovaprevir with either Cremophor or Lutrol 127, by 2 weeks crystalline material appeared. Impurity levels also increased, after 2 weeks with Cremophor or after 4 weeks with Lutrol 127. Upon further storage crystalline materials persisted.

TABLE 5
Crystal Formation and Impurity Levels of
Sovaprevir Plus Excipient Upon Storage

| | | XRPD Results/Impurities (HPLC) | | | |
|---|---|---|---|---|---|
| API + | Storage at | For Given Storage Time | | | |
| Excipient | Temp/RH | 0 Weeks | 2 Weeks | 4 Weeks | 8 Weeks |
| 1:1 API + | 40° C./ | A/0.095% | X/0.31% | X/0.42% | X/0.30% |
| Cremophor | 75% RH | | | | |
| 1:1 API + | 25° C./ | A/0.095% | X/0.16% | X/0.16% | X/NT |
| Cremophor | 60% RH | | | | |
| 1:1 API + | 40° C./ | A/0.080% | X/NT | X/0.36% | X/0.61% |
| Lutrol F127 | 75% RH | | | | |

A = Material amorphous in nature
X = Crystalline peaks seen
NT = Not tested

### Example 9

### Dissolution of Sovaprevir Tablets

TABLE 6 provides the dissolution profiles for a variety of Sovaprevir dosage forms. Sovaprevir dissolution is given as % dissolution by weight. USP method 36<711> is used to determine the dissolution profile of Sovaprevir tablets in 0.5% SLS at 37° C., except for the uncoated tablets which were tested using USP method 34<711>

TABLE 6
% Dissolution of Sovaprevir Tablets

| | |
|---|---|
| Tablet | Time (min) |

| Type | 10 | 20 | 30 | 45 | 60 | 75 |
|---|---|---|---|---|---|---|
| *100 mg | 69.8 | 89.4 | 94.8 | 97.8 | 99.1 | 100.1 |
| uncoated | | | | | | |
| *200 mg | 60.2 | 85.4 | 93.0 | 97.0 | 98.6 | 99.8 |
| uncoated | | | | | | |
| *200 mg | 67.2 | 90.1 | 97.3 | 100.9 | 102.2 | 103.2 |
| uncoated | | | | | | |
| 6 month | | | | | | |
| storage | | | | | | |
| 200 mg | 62.9 | 79.8 | 86.7 | 90.7 | 93.0 | 94.4 |
| coated | | | | | | |
| 03K | | | | | | |
| 200 mg | 56.3 | 75.3 | 83.9 | 89.9 | 92.4 | 94.6 |
| coated | | | | | | |
| 85F | | | | | | |
| 200 mg | 63.2 | 80.1 | 85.9 | 89.6 | 91.4 | 93.4 |
| coated | | | | | | |
| 85F | | | | | | |
| 6 month | | | | | | |
| storage | | | | | | |
| 300 mg | 43.3 | 64.6 | 75.5 | 83.5 | 87.0 | 90.8 |
| coated | | | | | | |
| 03K | | | | | | |
| 300 mg | 40.7 | 64.8 | 78.0 | 86.2 | 89.6 | 93.1 |
| coated | | | | | | |
| 85F | | | | | | |

*sample tested using USP 34<711>
Stored samples were maintained at 25° C./60% RH for the stated time before testing
03K refers to coating Opadry White 03K18416.
85F refers to coating Opadry White 85F18422.

### Example 10

### Stability Results for 100 MG Uncoated Sovaprevir Tablets

Stability data in TABLE 7 are for Sovaprevir tablets stored at a temperature of 25° C. and a relative humidity of 60%.

TABLE 7
Stability of 100 mg Uncoated Sovaprevir Tablets

| | Storage Time (Months) | | | | | | | | |
|---|---|---|---|---|---|---|---|---|---|
| | 0 | 1 | 2 | 3 | 6 | 9 | 12 | 18 | 24 |
| HPLC Assay (%) | 102.2 | 100.8 | 103.5 | 99.4 | 101.1 | 101.6 | 101.6 | 97.6 | 99.0 |
| Impurities (%) | 0.3 | 0.3 | 0.2 | 0.3 | 0.3 | 0.3 | 0.3 | 0.3 | 0.4 |
| Moisture (%) | 3.3 | 3.0 | 2.7 | 2.3 | 2.9 | 3.5 | 3.7 | 3.0 | 4.0 |
| Crystalline API Detected | ND | ND | ND | ND | ND | ND | ND | ND | ND |

ND = Not Detected

### Example 11

### Stability Results for 200 MG Uncoated Sovaprevir Tablets

Stability data in TABLE 8 are for Sovaprevir tablets stored at a temperature of 25° C. and a relative humidity of 60%.

TABLE 8
Stability of 200 mg Uncoated Sovaprevir Tablets

| | Storage Time (Months) | | | | | | | | |
|---|---|---|---|---|---|---|---|---|---|
| | 0 | 1 | 2 | 3 | 6 | 9 | 12 | 18 | 24 |
| HPLC Assay (%) | 103.7 | 103.7 | 100.9 | 104.1 | 104.0 | 102.2 | 102.2 | 99.6 | 102.9 |
| Impurities (%) | 0.3 | 0.3 | 0.3 | 0.4 | 0.3 | 0.3 | 0.3 | 0.3 | 0.4 |
| Moisture (%) | 3.7 | 3.2 | 2.7 | 2.4 | 2.9 | 3.5 | 3.9 | 3.0 | 3.9 |
| Crystalline API Detected | ND | ND | ND | ND | ND | ND | ND | ND | ND |

ND = Not Detected

### Example 12

### Stability Results for 200 MG Opadry White Coated Sovaprevir Tablets

Stability data in TABLE 9 are for Sovaprevir tablets stored at a temperature of 25° C. and a relative humidity of 60%.

TABLE 9
Stability of 200 mg 03K Coated Sovaprevir Tablets

| | Storage Time (Months) | | |
|---|---|---|---|
| | 0 | 1 | 3 |
| HPLC Assay (%) | 96.0 | 95.6 | 95.5 |
| Impurities (%) | 0.3 | 0.3 | 0.3 |
| Moisture (%) | 4.2 | 3.6 | 2.9 |

03K refers to coating: Opadry White 03K18416

### Example 13

### Stability Results for 300 MG Opadry White Coated Sovaprevir Tablets

Stability data in TABLE 10 are for Sovaprevir tablets stored at a temperature of 25° C. and a relative humidity of 60%.

TABLE 10
Stability of 300 mg 03K Coated Sovaprevir Tablets

| | Storage Time (Months) | | |
|---|---|---|---|
| | 0 | 1 | 3 |
| HPLC Assay (%) | 92.5 | 92.0 | 90.8 |
| Impurities (%) | 0.3 | 0.3 | 0.3 |
| Moisture (%) | 4.1 | 3.7 | 2.8 |

03K refers to coating Opadry White 03K18416

### Example 14

### Stability Results for 200 MG Opadry White Coated Sovaprevir Tablets

Stability data in TABLE 11 are for Sovaprevir tablets stored at a temperature of 25° C. and a relative humidity of 60%.

TABLE 11
Stability of 200 mg 85F Coated Sovaprevir Tablets

Storage Time (Months)

| | 0 | 1 | 3 | 6 |
|---|---|---|---|---|
| HPLC Assay (%) | 94.9 | 94.8 | 96.2 | 94.4 |
| Impurities (%) | 0.3 | 0.3 | 0.3 | 0.3 |
| Moisture (%) | 3.3 | 3.1 | 4.0 | 2.1 |

85F refers to coating Opadry White 85F18422

### Example 15

### Stability Results for 300 MG Opadry White Coated Sovaprevir Tablets

Stability data in TABLE 12 is for Sovaprevir tablets stored at a temperature of 25° C. and a relative humidity of 60%.

TABLE 12
Stability of 300 mg 85F Coated Sovaprevir Tablets

Storage Time (Months)

| | 0 | 1 | 3 | 6 |
|---|---|---|---|---|
| HPLC Assay (%) | 93.4 | 91.2 | 93.1 | 91.9 |
| Impurities (%) | 0.3 | 0.3 | 0.3 | 0.3 |
| Moisture (%) | 3.6 | 3.0 | 3.5 | 2.1 |

85F refers to coating Opadry White 85F18422

What is claimed is:

1. A Sovaprevir immediate release composition, comprising an amount of Sovaprevir from about 100 mg to about 400 mg Sovaprevir; wherein the composition provides
an AUC₀-₇₂ of about 150 ng·hr/ml to about 2500 ng·hr/ml;
a mean Cₘₐₓ of about 20 ng/ml to about 750 ng/ml; and
a mean Tₘₐₓ of about 0.5 to about 5.0 hr.

2. A 200 mg Sovaprevir composition of claim 1, providing
an AUC₀-₇₂ of about 300 ng·hr/ml to about 700 ng·hr/ml
a mean Cₘₐₓ of about 40 ng/ml to about 170 ng/ml; and
a mean Tₘₐₓ of about 1.5 to about 3.7 hr.

3. A Sovaprevir tablet core, comprising Sovaprevir and a crystal growth inhibitor selected from hydroxypropyl methyl cellulose (HPMC), HPC (hydroxypropyl cellulose), hypromellose acetate succinate (HPMCAS), polyvinyl pyrrolidone (PVP), and copovidone (PVP-VA), or any combination of the foregoing, wherein ratio of Sovaprevir to the crystal growth inhibitor is from about 40:60 (w/w) to about 60:40 (w/w).

4. The Sovaprevir tablet core of claim 3, wherein the crystal growth inhibitor is HPMC and the ratio of Sovaprevir to crystal growth inhibitor is about 50:50 (w/w).

5. The Sovaprevir tablet core of claim 3, wherein the crystal growth inhibitor is a combination of HPMC and a second crystal growth inhibitor and the ratio of Sovaprevir to crystal growth inhibitor is about 50:50 (w/w).

6. The Sovaprevir tablet core of claim 3, wherein the tablet core also comprises a filler, a disintegrant, and a lubricant.

7. The Sovaprevir tablet core of claim 6 wherein the disintegrant is croscarmellose sodium, crospovidone, sodium starch glycolate, or a combination of the foregoing.

8. The Sovaprevir tablet core of claim 6, wherein the lubricant is calcium stearate, magnesium stearate, glyceryl monostearate, glyceryl behenate, stearic acid, mineral oil, talc, or a combination of the foregoing.

9. The Sovaprevir tablet core of claim 8, wherein the filler is microcrystalline cellulose, silicified microcrystalline cellulose, ethyl cellulose, lactose, or a combination of the foregoing.

10. The Sovaprevir tablet core of claim 6, wherein the filler is microcrystalline cellulose or silicified microcrystalline cellulose or a combination thereof, the disintegrant is croscarmellose sodium, and the lubricant is magnesium stearate.

11. The Sovaprevir tablet core of claim 10, wherein the tablet core comprises about 20% to about 30% (weight %) Sovaprevir, about 20% to about 30% HPMC, about 40% to about 60% silicified microcrystalline cellulose, about 1% to about 10% croscarmellose sodium, and about 0.1% to about 1% magnesium stearate.

12. A coated Sovaprevir tablet comprising the Sovaprevir tablet core of claim 3 and an immediate release coating.

13. The Sovaprevir tablet core of claim 6, wherein the tablet core exhibits a dissolution profile after combining the tablet with 900 mL of 0.5% SLS in DI water at 37° C. according to USP 36 <711> (paddle) at a speed of 75 rpm, wherein at least 60% of the total amount of Sovaprevir is released after 10 minutes.

14. The coated Sovaprevir tablet of claim 12, wherein the tablet core exhibits a dissolution profile after combining the tablet with 900 mL of 0.5% SLS in DI water at 37° C. according to USP 36 <711> (paddle) at a speed of 75 rpm, wherein at least 40% of the Sovaprevir is released after 10 minutes.

15. The Sovaprevir tablet core of claim 13, wherein 90% of the total amount of Sovaprevir is released after 30 minutes.

16. The coated Sovaprevir tablet of claim 12, wherein the tablet exhibits a dissolution profile after combining the tablet with 900 mL of 0.5% SLS in DI water at 37° C. according to USP 36 <711> (paddle) set at 75 rpm, wherein at least 70% of the total amount of Sovaprevir is released after 30 minutes.

17. The Sovaprevir tablet core of claim 15, wherein at least 95% of the total amount of Sovaprevir released after 60 minutes.

18. The Sovaprevir tablet of claim 16, wherein at least 90% of the Sovaprevir is released after 60 minutes.
